(19) United States
(12) Patent Application Publication
(10) Pub. No.: US 2026/0242846 A1
(43) Pub. Date: Aug. 20, 2026

(54) KINASE ACTIVITY EVALUATION APPARATUS AND NON-TRANSITORY COMPUTER READABLE STORAGE MEDIUM

(71) Applicant: CANON MEDICAL SYSTEMS CORPORATION, Otawara-shi (JP)

(72) Inventor: Hiroto SATAKE, Brighton, MA (US)

(73) Assignee: CANON MEDICAL SYSTEMS CORPORATION, Otawara-shi (JP)

(21) Appl. No.: 19/542,420

(22) Filed: Feb. 17, 2026

(30) Foreign Application Priority Data
Feb. 18, 2025 (JP) 2025-024492
Feb. 17, 2026 (JP) 2026-023528

(51) Int. Cl. C12Q 1/48

(52) U.S. Cl. CPC C12Q 1/485

(57) ABSTRACT

An apparatus for assessing kinase activity according to an embodiment, comprises processing circuitry configured to measure an amount of phosphorylated amino acid in immobilized substrate protein generated when the substrate protein is reacted with a kinase to be evaluated, as an amount of phosphorylated amino acid in a specimen; read, from a memory, information which links reaction time, an amount of immobilized substrate protein having autophosphorylation activity, and an amount of autophosphorylated amino acid generated in the substrate protein having autophosphorylation activity and correct the amount of phosphorylated amino acid in a specimen based on a measurement result for the specimen and the information; and evaluate kinase activity based on the corrected amount of phosphorylated amino acid in the specimen.

### CROSS REFERENCE TO RELATED APPLICATIONS

This application is based upon and claims the benefit of priority from the prior Japanese Patent Application No. 2025-024492, filed on Feb. 18, 2025, the entire contents of which are incorporated herein by reference.

### FIELD

The embodiments disclosed in this description and the drawings relate to a kinase activity evaluation apparatus and non-transitory computer readable storage medium.

### BACKGROUND

In cells, there are molecular mechanisms for transmitting information to express various functions. Posttranslational modification of proteins, in particular, phosphorylation of proteins, plays an important role in signal transduction of the cells. The intracellular signal transduction mechanism forms a complicated network in which a large number of molecules work together. It is considered that, signal transduction abnormality caused by mutation of a protein phosphorylation enzyme, i.e., protein kinase, or activation due to overexpression thereof is involved in many diseases.

In this context, techniques for evaluating kinase activity have been developed and put into use. As a means for evaluating the activity of a kinase contained in a specimen, e.g., a technique for estimating and evaluating the activity of a kinase based on the measurement amount of phosphorylated amino acid (tyrosine, serine, threonine) generated in a spot containing a substrate protein immobilized on a substrate, when the spot is reacted with the specimen.

However, it is known that some of the substrate proteins serve as a kinase and phosphorylate themselves; that is, perform “autophosphorylation”. When kinase activity of a specimen is evaluated based on the amount of phosphorylated amino acid generated in a spot containing a substrate protein as an index, if the phosphorylated amino acid generated through autophosphorylation is present as background, accurate quantification of phosphorylated amino acid generated in the spot by a kinase of the specimen is inhibited, with the result that accurate evaluation of kinase activity may become difficult.

### BRIEF DESCRIPTION OF THE DRAWINGS

FIG. 1 is a block diagram showing the functional configuration of a kinase activity evaluation apparatus according to a first embodiment;

FIG. 2 is a flowchart of a kinase activity evaluation process performed by the kinase activity evaluation apparatus according to the first embodiment;

FIG. 3 shows examples of data to be stored as reference data in the memory unit: a graph (left) showing the distribution of the amount of immobilized substrate (I680 nm) and the amount of phosphorylated tyrosine (I790 nm) due to autophosphorylation acquired for a substrate protein (e.g., BMX), and fitting results; and a graph (right) showing the method for acquiring the fluorescence intensity ratio γ of the fluorescence intensity at I680 nm per substrate molecule and of the fluorescence intensity at I790 nm per phosphorylated tyrosine site;

FIG. 4 shows examples of data to be stored as reference data in the memory unit, that is, the number α of phosphorylation sites, maximum reaction rate Vmax_{self}, Michaelis constant Km_{self} of an autophosphorylated substrate protein (e.g., BMX), and shows a graph showing distribution data of the amount of immobilized substrate and the amount of autophosphorylated amino acid at each phosphorylation reaction time T estimated based on the data;

FIG. 5 is a graph showing the results obtained by fitting the acquired data in accordance with the Michaelis-Menten equation for a phosphorylation reaction including a heterophosphorylation reaction (phosphorylation reaction caused by a kinase derived from a specimen) and an autophosphorylation reaction;

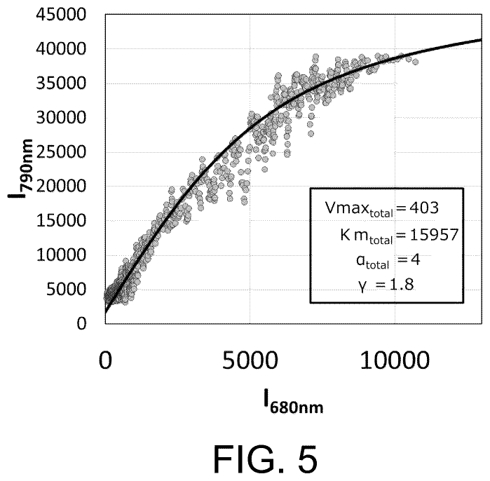

FIG. 6 includes graphs showing a correction method (correction method 1) for estimating the distribution of phosphorylated amino acid due to a heterophosphorylation reaction by taking the difference between the distribution of the amount of phosphorylated amino acid due to both a heterophosphorylation reaction and an autophosphorylation reaction obtained for a substrate and the distribution of the amount of phosphorylated amino acid due to the autophosphorylation reaction for the substrate obtained from the memory unit by reference;

FIG. 7 is a graph showing the amount of phosphorylated amino acid obtained by a correction method (correction method 2) for estimating the distribution of phosphorylated amino acid due to a heterophosphorylation reaction obtained by fitting acquired data (distribution of the amount of immobilized substrate and the amount of phosphorylated amino acid including a heterophosphorylation reaction and an autophosphorylation reaction acquired for a substrate) based on the fitting equation which takes competition between a heterophosphorylation reaction and an autophosphorylation reaction, into consideration and based on information which links the amount of immobilized substrate and the amount of amino acid due to autophosphorylation and which is obtained from the memory unit by reference;

FIG. 8 is a graph showing comparison results between correction data (correction method 2) which takes competition between a heterophosphorylation reaction and an autophosphorylation reaction into consideration and correction data (correction method 1) which does not take competition between a heterophosphorylation reaction and an autophosphorylation reaction into consideration.

### DETAILED DESCRIPTION

Hereinafter, embodiments of a kinase activity evaluation apparatus and non-transitory computer readable storage medium will be described in detail with reference to the drawings.

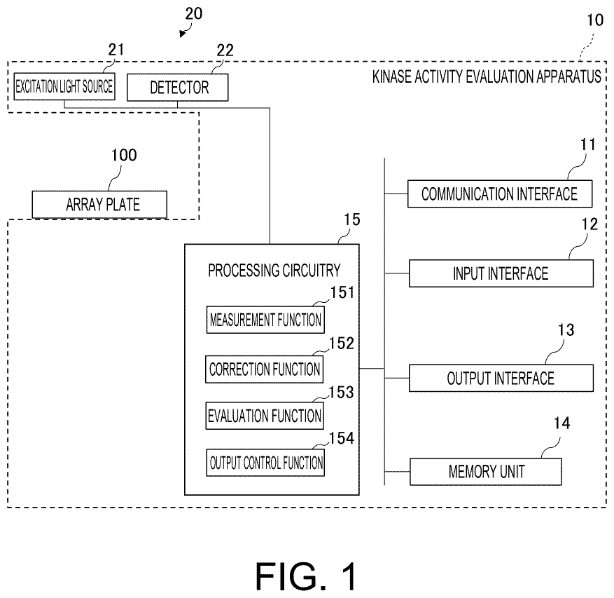

FIG. 1 is a block diagram showing the functional configuration of a kinase activity evaluation apparatus according to a first embodiment. A kinase activity evaluation apparatus 10 according to the first embodiment is an apparatus for measuring the amount of phosphorylated amino acid generated in immobilized substrate protein when the substrate protein is reacted with the kinase to be evaluated, and evaluating the activity of the kinase based on the amount of phosphorylated amino acid.

The “substrate protein” in the kinase activity evaluation apparatus 10 according to the first embodiment is a single or a plurality of proteins to be phosphorylated by the kinase to be evaluated for activity, and immobilized on an arbitrary carrier or optionally contained in a spot. The “substrate protein” is not particularly limited, and can be appropriately determined according to the purpose, and may be naturally derived, synthesized, or genetically modified. This “substrate protein” may be defined as follows. All types of substrate proteins have autophosphorylation activity; some types of substrate proteins species have autophosphorylation activity, but at least a part of substrate proteins have autophosphorylation activity. Herein, the phrase that a substrate protein has autophosphorylation activity means that the substrate protein has the ability to phosphorylate itself or the same type of substrate protein coexisting therewith.

In the kinase activity evaluation apparatus 10 according to the first embodiment, the carrier on which a substrate protein is to be immobilized is not particularly limited, but an appropriate substrate may be employed as the carrier. Examples of the substrate include, but are not particularly limited to, a two-dimensional plate, a polymer gel, a fiber and a fiber sheet, a bead, and a rod. The surface of the “substrate” may be smooth, but may have a micro/nanostructure such as a porous structure or a fiber. The “substrate” is preferably a two-dimensional plate. Examples of the two-dimensional plate includes plate-like substrates such as a slide glass and a cover glass, and well substrates such as an array plate. The “substrate” is more preferably a well substrate such as an array plate. The array plate has spots containing a substrate protein and used for comprehensive analysis of a specimen, and is also called, for example, a microchip, a microarray, or a protein chip.

In the kinase activity evaluation apparatus 10 according to the first embodiment, the substrate protein may be immobilized on a carrier by direct binding or binding with a linker substance interposed between them, or immobilized in a state of being engulfed in a gel substance. The substrate protein may be physically or chemically immobilized on a carrier.

The “spot” in the kinase activity evaluation apparatus 10 according to the first embodiment means a redetermined closed region on a carrier. The form of the “spot” is not particularly limited, and may be, for example, a square, a rectangle, a circle, or an ellipse, but is preferably a circle.

The “specimen” containing the kinase to be evaluated in the kinase activity evaluation apparatus 10 according to the first embodiment can be appropriately determined according to the purpose by the person who carries out the method. Any specimen may be used as long as it contains a kinase. Examples of the specimen include a biologically derived substance, an extract from, e.g., a living body, blood, a blood-derived substance, a food, a food-derived substance, a natural product, a natural product-derived substance, a culture solution-derived substance. The specimen may be appropriately treated in advance according to the purpose or procedure, or a reagent may be added to the specimen in advance. The specimen can be present in a gaseous form, a solid form, or a liquid form, and is appropriately diluted, suspended, or extracted in water, physiological saline, a buffer solution, or another solution and used in a liquid form. The specimen may contain a preservative and other additives. In addition, a reagent varied according to the purpose is added to the specimen.

In the kinase activity evaluation apparatus 10 according to the first embodiment, the measurement of the amount of phosphorylated amino acid generated in the substrate protein can be performed, for example, by bringing a specimen into contact with a spot on a carrier, thereby performing a phosphorylation reaction between the kinase contained in the specimen and the substrate protein contained in the spot to generate phosphorylated amino acid in the substrate protein, and quantifying the amount of the generated phosphorylated amino acid. The amount of the generated phosphorylated amino acid may be directly quantified, or indirectly quantified, for example, by further labeling the phosphorylated substrate protein to generate labeled phosphorylated amino acid in the phosphorylated substrate protein, and performing quantification based on the signal information derived from the labeling agent.

The phrase that further labeling the phosphorylated substrate protein means introducing a labeling agent into the phosphorylated substrate protein via a phosphorylation site recognition substance. The phosphorylation site recognition substance refers to a substance that specifically recognizes a phosphorylated site in a substrate protein phosphorylated by a phosphorylating enzyme. Examples of the phosphorylation site recognition substance include, but are not particularly limited to, an antiphosphorylated amino acid antibody. When the labeling agent is not bound to the phosphorylation site recognition substance, a labeling agent may be further introduced through a secondary antibody or a specific reaction including a biotin avidin reaction.

Examples of the labeling agent include radioactive substances, enzymes, capture molecules, fluorescent substances, luminescent substances, and metal particles, and preferably optically detectable substances. Examples of such optically detectable substances include fluorescent substances, chemiluminescent substances, phosphorescent substances, dyes, gold nanoparticles, fluorescent particles, enzymes for chemiluminescence or color reactions, and fine particles having absorption at a predetermined wavelength. The labeling agent may contain an antibody, a ligand, or another binding site. The binding or introduction of the labeling agent includes binding the labeling agent by a hydrophobic interaction, an electrostatic interaction, a van der Waals interaction, a hydrogen bond, or a covalent bond and introducing the labeling agent during synthesis. Besides these, known labelling methods may be included.

The amount of phosphorylated amino acid of the labeled phosphorylated substrate protein is quantified based on the signal information derived from the labeling agent. Based on the intensity of a signal, information on the amount of the labeled phosphorylated substrate protein can be obtained. In other words, the amount of phosphorylated amino acid of the labeled phosphorylated substrate protein can be quantified. Such a signal is preferably an optically detectable signal such as light intensity information from a spot and spectral information.

When the labeling agent is a fluorescent substance, the optical system can be used without limitation as long as it can excite the fluorescent substance and detect fluorescence. In the case shown in FIG. 1, the kinase activity evaluation apparatus 10 has an optical system 20. In order to excite a fluorescent substance, an excitation light source 21 can be used in the optical system 20. Examples of such an excitation light source include a laser light source, a light emitting diode, a mercury arc lamp, and a tungsten halogen lamp. For detection, a detector 22 such as a CCD camera or a photodiode may be used. The optical system 20 appropriately contains a filter to emit or detect light having a predetermined wavelength. Furthermore, the optical system 20 may have a lens. The optical system 20 may be a scanning type or a non-scanning type. As the optical system, for example, a confocal optical unit can be used. Note that, in the case shown in FIG. 1, the optical system 20 is integrated with the kinase activity evaluation apparatus 10, but is not limited to this. The optical system 20 may not be integrated with the kinase activity evaluation apparatus 10. In the following description, the case where the optical system 20 integrated with the kinase activity evaluation apparatus 10 will be described.

When chemiluminescence or a dye is used as the labeling agent, the excitation light source may not be provided.

Furthermore, as illustrated in FIG. 1, the kinase activity evaluation apparatus 10 has a communication interface 11, an input interface 12, an output interface 13, a memory unit 14, and a processing circuitry 15, together with the optical system 20.

The communication interface 11 implements communication protocols according to the network configuration. The communication interface 11 conducts communication with other devices via the network according to the communication protocols.

The input interface 12 is a circuit that receives inputs from the user. The input interface 12 is actually constituted of, for example, a mouse, a keyboard, a trackball, a manual switch, a foot switch, a button, and a joystick.

The output interface 13 outputs images and information. For example, the output interface 13 outputs, e.g., evaluation results of kinase activity by an evaluation function 154 (described later) and a graphical user interface (GUI) for receiving operations from the user. The output interface 13 is constituted of, for example, a liquid crystal display, a cathode ray tube (CRT) display, and a speaker. The output interface 13 corresponds to an output unit in this embodiment.

The memory unit 14 is actually constituted of, for example, a semiconductor memory device such as random access memory (RAM) and a flash memory, a hard disk, or an optical disk. In this embodiment, the memory unit 14 stores, for example, “Information that links the reaction time of the substrate protein having autophosphorylation activity, the amount of immobilized substrate protein having autophosphorylation activity, and the amount of autophosphorylated amino acid of the substrate protein having autophosphorylation activity”.

Examples of the “information that links the reaction time of the substrate protein having autophosphorylation activity, the amount of immobilized substrate protein having autophosphorylation activity, and the amount of autophosphorylated amino acid of the substrate protein having autophosphorylation activity” (hereinafter, sometimes referred to as “linking information”. The linking information is the example of the information.) and stored in the memory unit 14 of the kinase activity evaluation apparatus 10 according to the first embodiment include, but are not particularly limited to, reaction time when a substrate protein having autophosphorylation activity is subjected to an autophosphorylation reaction, the value of the amount of immobilized substrate protein having autophosphorylation activity, the value of the amount of autophosphorylated amino acid of the substrate protein having autophosphorylation activity per reaction time, and the number of effective phosphorylation sites, maximum reaction rate (Vmax), and Michaelis constant (Km) (these values may be either actually measured values or known values) of the substrate protein having autophosphorylation activity; and preferably include 1 or 2 or more selected from the group consisting of the number of effective phosphorylation sites, maximum reaction rate (Vmax), and Michaelis constant (Km) of the substrate protein having autophosphorylation activity.

The number of phosphorylation sites (sites to be phosphorylated) of a substrate protein stored in the memory unit 14 of the kinase activity evaluation apparatus 10 according to the first embodiment may vary depending on the type of substrate protein. In the specification, the number of phosphorylation sites per molecule of a predetermined substrate protein is defined as the number of effective phosphorylation sites of the substrate protein.

The phosphorylation sites of a substrate protein having autophosphorylation activity can be classified into two categories, that is, “a phosphorylation site that undergoes not only a heterophosphorylation reaction (phosphorylation reaction caused by a kinase other than a substrate protein itself having autophosphorylation activity, such as a kinase derived from a specimen) but also an autophosphorylation reaction”, and “a phosphorylation site that undergoes a heterophosphorylation reaction and does not undergo an “autophosphorylation reaction”. The number of effective phosphorylation sites, which corresponds to the substrate protein having autophosphorylation activity and which is stored in the memory unit 14 of the kinase activity evaluation apparatus 10 according to the first embodiment preferably includes, but is not limited to, the number of all effective phosphorylation sites (“phosphorylation sites that undergo not only a heterophosphorylation reaction but also an autophosphorylation reaction” and “phosphorylation sites that undergoes a heterophosphorylation reaction and does not undergo an “autophosphorylation reaction”) of a substrate protein having autophosphorylation activity or the number of “phosphorylation sites that undergo not only a heterophosphorylation reaction but also an autophosphorylation reaction”.

In the specification, the number of all effective phosphorylation sites of a substrate protein having autophosphorylation activity is sometimes referred to as “αₜₒₜₐₗ”. Of them, the number of phosphorylation sites that undergo not only a heterophosphorylation reaction but also an autophosphorylation reaction is sometimes referred to as “α”.

The number of effective phosphorylation sites of a predetermined substrate protein is not particularly limited, but data directly acquired by, e.g., calculation, may be added to the memory unit. Also, the data may be stored in the memory unit, and can be used as the value of the number of effective phosphorylation sites of a predetermined substrate protein as necessary when another evaluation is made.

If a predetermined value is stored as the number of effective phosphorylation sites to a predetermined substrate protein in the memory unit, the value may be used by reference. In this case, when the number of effective phosphorylation sites of a predetermined substrate protein is known, the value may be used by reference. For example, the value identified by the PhosphoSitePlus online tool may be used by reference. In this case, it is preferable that the value corresponding to the same type of kinase as the kinase to be evaluated is preferably employed as the number of effective phosphorylation sites of a predetermined substrate protein, but the value corresponding to a similar type of kinase to the kinase to be evaluated may be employed as the number of effective phosphorylation sites of the predetermined substrate protein.

The number of effective phosphorylation sites of a predetermined substrate protein and stored in the memory unit 14 of the kinase activity evaluation apparatus 10 according to the first embodiment is preferably determined depending on the combination of the substrate protein and a kinase.

The maximum reaction rate (Vmax) of the phosphorylation reaction of a substrate protein having autophosphorylation activity and stored in the memory unit 14 of the kinase activity evaluation apparatus 10 according to the first embodiment preferably includes, but is not limited to, the maximum reaction rate (Vmax_{self}) of the autophosphorylation reaction at the phosphorylation site of the substrate protein having autophosphorylation activity that undergoes not a only heterophosphorylation reaction but also an autophosphorylation reaction. The maximum reaction rate of the phosphorylation reaction of the substrate protein having autophosphorylation activity stored in the memory unit 14 of the kinase activity evaluation apparatus 10 according to the first embodiment may be an actually measured value, or if a predetermined value is present, the predetermined value may be used by reference.

In the specification, the maximum reaction rate of the phosphorylation reaction that occurs irrespective of autophosphorylation and heterophosphorylation at all effective phosphorylation sites of a substrate protein having autophosphorylation activity is sometimes referred to as “Vmaxₜₒₜₐₗ”, and the maximum reaction rate of the heterophosphorylation reaction at all effective phosphorylation sites of a substrate protein is sometimes referred to as “Vmaxₒₜₕₑᵣ”.

The Michaelis constant (Km) of the phosphorylation reaction of a substrate protein having autophosphorylation activity and stored in the memory unit 14 of the kinase activity evaluation apparatus 10 according to the first embodiment preferably includes, but is not limited to, the Michaelis constant (Km_{self}) of the autophosphorylation reaction at the phosphorylation site that undergoes not only a heterophosphorylation reaction but also an autophosphorylation reaction of the substrate protein having autophosphorylation activity. The Michaelis constant of the phosphorylation reaction of a substrate protein having autophosphorylation activity stored in the memory unit 14 of the kinase activity evaluation apparatus 10 according to the first embodiment may be an actually measured value, or if a predetermined value is present, the predetermined value may be used by reference.

In the specification, the Michaelis constant of a phosphorylation reaction that occurs irrespective of autophosphorylation and heterophosphorylation at all effective phosphorylation sites of a substrate protein having autophosphorylation activity is sometimes referred to as “Kmₜₒₜₐₗ”, whereas the Michaelis constant of a phosphorylation reaction caused by a heterophosphorylation reaction at all effective phosphorylation sites of a substrate protein having autophosphorylation activity is sometimes referred to as “Kmₒₜₕₑᵣ”.

The value of a maximum reaction rate of the phosphorylation reaction of predetermined substrate protein can be calculated based on, for example, the result obtained by plotting the value of the amount of phosphorylated amino acid of a substrate protein in a predetermined region on a spot as a function of the value of the amount of the substrate protein immobilized in the region, but at this time, the relationship between these values may be fitted in accordance with the Michaelis-Menten equation. When fitting was made in accordance with the Michaelis-Menten equation, the Michaelis constant can be further calculated. Note that, instead of fitting in accordance with the Michaelis-Menten equation, the equation (Lineweaver-Burk equation) obtained by taking the reciprocal of the Michaelis-Menten equation may be used. The methods for calculating the maximum reaction rate and the Michaelis constant are not particularly limited, and the maximum reaction rate and the Michaelis constant can be calculated by known methods. In addition, these data may be stored in the memory unit 14, and can be used as the values of the maximum reaction rate and Michaelis constant of the phosphorylation reaction of a predetermined substrate protein as necessary when another evaluation is made.

Returning to FIG. 1, the processing circuitry 15 is an arithmetic circuit performing various operations. The processing circuitry 15 is constituted of, for example, a processor. In the processing circuitry 15 according to this embodiment, kinase activity is evaluated based on, for example, the amount of phosphorylated amino acid in a specimen.

As illustrated in FIG. 1, the processing circuitry 15 has a measurement function 151, a correction function 152, an evaluation function 153, and an output control function 154. The measurement function 151 corresponds to a measurement unit according to this embodiment; the correction function 152 corresponds to a correction unit according to this embodiment; the evaluation function 153 corresponds to an evaluation unit according to this embodiment; and the output control function 154 corresponds to an output control unit according to this embodiment.

In the embodiment illustrated in FIG. 1, each of the processing functions performed by the measurement function 151, the correction function 152, the evaluation function 153, and the output control function 154 of the processing circuitry 15 are stored in the memory unit 14 in the form of a program executable by a computer. The processing circuitry 15 is a processor that executes a function corresponding to each of the programs by reading the program from the memory unit 14. In other words, the processing circuitry 15 just after reading each of the programs has individual functions illustrated in the processing circuitry 15 of FIG. 1. Note that, in FIG. 1, it has been described that the measurement function 151, the correction function 152, the evaluation function 153, and the output control function 154 are executed by the single processing circuitry 15. However, these functions may be executed by using a plurality of independent processors in combination to constitute the processing circuitry 15 and executing the programs by individual processors.

The measurement function 151 measures the amount of phosphorylated amino acid generated in the immobilized substrate protein when the substrate protein is reacted with the kinase to be evaluated, as the amount of phosphorylated amino acid in a specimen. Specifically, the measurement function 151 controls an optical system to excite a fluorescent substance introduced into the phosphorylated substrate protein as a labeling agent by an excitation light source of the optical system, and detects fluorescence by a detector to measure the amount of phosphorylated amino acid generated in the immobilized substrate protein when the substrate protein is reacted with the kinase to be evaluated.

The correction function 152 reads “linking information” from the memory unit 14, and corrects the amount of phosphorylated amino acid in a specimen based on the measurement result for the specimen and the “linking information”. The correction method is not particularly limited, and can be performed, for example, by correcting the distribution of the amount of the phosphorylated amino acid of a substrate protein reacted with the kinase to be evaluated relative to the immobilized amount of a substrate protein based on the reaction time and the amount of the autophosphorylated amino acid relative to the amount of the substrate protein, or correcting the amount of the phosphorylated amino acid of a substrate protein reacted with the kinase to be evaluated relative to the immobilized amount of a substrate protein based on the amount of the autophosphorylated amino acid at reaction time T, which is estimated from the number of effective substrate sites, maximum reaction rate and Michaelis constant of the autophosphorylation reaction of a substrate protein. In this embodiment, the correction function 152 corrects the amount of phosphorylated amino acid in a specimen by subtracting or removing the amount of autophosphorylated amino acid from the amount of phosphorylated amino acid in the specimen, as a measurement result for the specimen.

The method for acquiring the amount of immobilized substrate protein is not particularly limited. The amount of immobilized substrate protein can be acquired by, for example, quantification by a surface plasmon resonance (SPR) method, quantification based on electrochemical characteristics (for example, potential, current value, impedance, and capacitance), quantification based on the distribution of existing elements acquired by, e.g., X-ray spectroscopy, quantification using an AFM, or quantification based on the signal information derived from a labeling agent introduced into the substrate. When the value of the amount of immobilized substrate protein is known, the value may be used.

The evaluation function 153 evaluates kinase activity based on the amount of phosphorylated amino acid in a specimen corrected by the correction function 152.

The output control function 154 controls the output interface 13 so as to output the evaluation result of the kinase activity by the evaluation function 153.

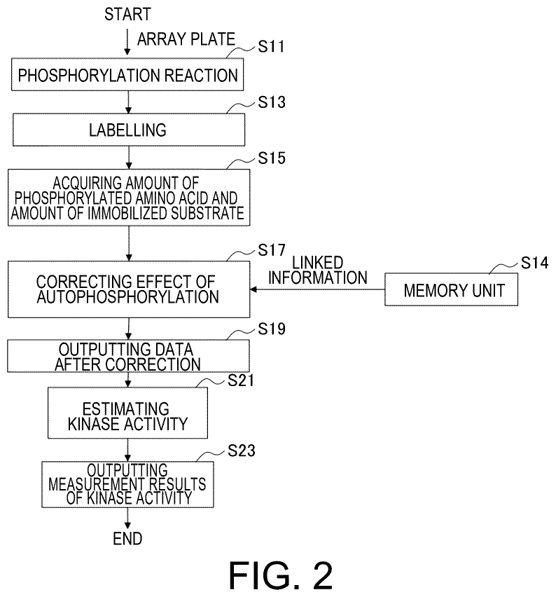

FIG. 2 is a flowchart for evaluating kinase activity by the kinase activity evaluation apparatus 10 according to the first embodiment.

As illustrated in FIG. 2, first, the kinase activity evaluation apparatus 10 stands by until the user executes the phosphorylation reaction of a kinase and a substrate protein on the array plate 100 (step S11). That is, the user brings a specimen into contact with a spot on a carrier to execute the phosphorylation reaction of a kinase contained in the specimen and a substrate protein contained in the spot. As a result, a phosphorylated substrate protein can be obtained.

Next, as illustrated in FIG. 2, the kinase activity evaluation apparatus 10 stands by until the phosphorylated substrate protein is further labeled by the user (step S13). That is, the user introduces a fluorescent substance as a labeling agent into the phosphorylated substrate protein via the phosphorylation site recognition substance. As a result, a labeled phosphorylated substrate protein can be obtained.

Next, as illustrated in FIG. 2, the measurement function 151 in the processing circuitry 15 of the kinase activity evaluation apparatus 10 acquires the amount of phosphorylated amino acid and the amount of immobilized substrate (step S15). Specifically, the measurement function 151 acquires the amount of phosphorylated amino acid of the labeled phosphorylated substrate protein and the amount of immobilized substrate protein (substrate). More specifically, the measurement function 151 controls the excitation light source 21 to apply excitation light to the labeled phosphorylated substrate protein of the array plate 100 installed in the kinase activity evaluation apparatus 10, and detects the fluorescence of the labeled phosphorylated substrate protein by the detector 22, thereby measuring the amount of phosphorylated amino acid in the specimen and the signal derived from the labeling agent introduced into the substrate at a point where the substrate protein is immobilized. As described above, the measurement function 151 acquires the amount of phosphorylated amino acid and the amount of immobilized substrate protein (substrate). Then, the measurement function 151 measures the amount of the phosphorylated amino acid and the amount of immobilized substrate protein (substrate) at individual points where the substrate protein is immobilized, thereby acquiring the distribution of the amount of the phosphorylated amino acid and the amount of immobilized substrate protein (substrate). Note that, the point at which the distribution of the amount of phosphorylated amino acid and the amount of immobilized substrate protein (substrate) is acquired is desirably a region having a smaller diameter than that of the spot.

Next, as illustrated in FIG. 2, the correction function 152 in the processing circuitry 15 of the kinase activity evaluation 10 corrects the effect of autophosphorylation (step S17). Specifically, the correction function 152
reads “linking information” from the memory unit 14, and corrects the effect of autophosphorylation of the substrate protein based on the amount of phosphorylated amino acid, the amount of immobilized substrate acquired in step S15 and the “linking information”. More specifically, the correction function 152 reads “linking information” including the reaction time and the amount of autophosphorylated amino acid relative to the amount of a substrate protein from the memory unit 14, and subtracts or removes the amount of the autophosphorylated amino acid contained in the “linking information” from the amount of the phosphorylated amino acid in the specimen acquired in step S15, thereby correcting the amount of the phosphorylated amino acid in a specimen. That is, the correction function 152 corrects the amount of the phosphorylated amino acid in the specimen, thereby calculating the amount of the heterophosphorylated amino acid.

Next, as illustrated in FIG. 2, the correction function 152 outputs the corrected data (step S19). Specifically, the correction function 152 outputs the corrected amount of phosphorylated amino acid in a specimen to the evaluation function 153 as the corrected data. Note that, in step S19, the correction function 152 outputs the corrected amount of phosphorylated amino acid in the specimen to the evaluation function 153, but the output destination of the corrected amount of phosphorylated amino acid in the specimen is not limited to the evaluation function 153. That is, the output destination of the corrected amount of phosphorylated amino acid in the specimen is arbitrarily selected and the corrected amount of phosphorylated amino acid in the specimen may be output to the output interface 13.

Next, as illustrated in FIG. 2, the evaluation function 153 in the processing circuitry 15 of the kinase activity evaluation apparatus 10 estimates kinase activity (step S21). Specifically, the evaluation function 153 estimates the activity of a kinase based on the corrected amount of phosphorylated amino acid in the specimen, thereby evaluating the activity of the kinase.

Next, as illustrated in FIG. 2, the output control function 154 in the processing circuitry 15 of the kinase activity evaluation apparatus 10 outputs the estimation result of kinase activity (step S23). Specifically, the output control function 154 controls the output interface 13 so as to output the evaluation result of the kinase activity evaluated in step S21.

When step S23 is executed, the kinase activity evaluation is terminated.

As described above, the kinase activity evaluation apparatus 10 according to the first embodiment measures the amount of phosphorylated amino acid generated in immobilized substrate protein when the substrate protein is reacted with the kinase to be evaluated, as an amount of phosphorylated amino acid in a specimen, reads linking information from the memory unit 14, corrects the amount of phosphorylated amino acid in the specimen based on the measurement result for the specimen and the information, and evaluates the kinase activity based on the corrected amount of phosphorylated amino acid in the specimen. Thus, it is possible to evaluate the kinase activity from which the effect of the autophosphorylation reaction of the substrate protein itself is corrected.

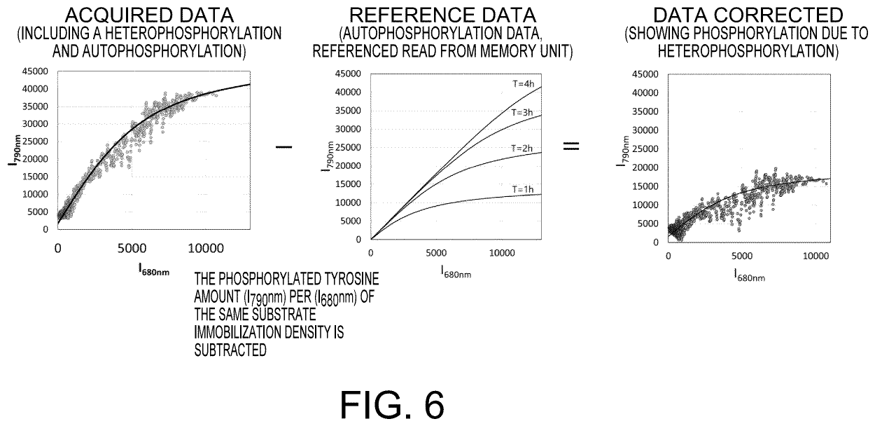

Note that, in the kinase activity evaluation apparatus 10 according to the first embodiment described above, the “linking information” may include the number of effective substrate sites, maximum reaction rate, and Michaelis constant of the autophosphorylation reaction of a substrate protein. In this case, in step S17 of the kinase activity evaluation, the correction function 152 may read “linking information” including the number of effective substrate sites, maximum reaction rate, and Michaelis constant of the autophosphorylation reaction of a substrate protein, and estimate the amount of autophosphorylated amino acid at reaction time T based on the number of effective substrate sites, maximum reaction rate, and Michaelis constant of the autophosphorylation reaction of a substrate protein, as illustrated in FIG. 6. The estimated amount of autophosphorylated amino acid is subtracted or removed from the amount of phosphorylated amino acid depending on the amount of immobilized substrate in the specimen acquired in step S15. In this manner, the corrected amount of phosphorylated amino acid in the specimen may be calculated.

### Examples

The present invention will be specifically described based on the following examples, but the present invention is not limited to these examples. The content is represented by % by mass unless otherwise specified.

### Method for Acquiring Reference Data on Autophosphorylation

In this Example, a method for acquiring reference data on autophosphorylation of a substrate protein having autophosphorylation activity and included in the “information” stored in a memory unit will be described. Herein, taking BMX (a type of non-receptor tyrosine kinase, molecular weight: 78011 Da) as an example of a substrate protein having autophosphorylation activity, a method for acquiring reference data of autophosphorylation in a state where BMX is immobilized on a substrate as a spot will be described.

To a glutathione (GSH) coated slide glass, which was prepared by a method according to the method described in Non-Patent Document 1 (Tadashi Manabe et al., “IGF2 Autocrine-Mediated IGF1R Activation Is a Clinically Relevant Mechanism of Osimertinib Resistance in Lung Cancer”, Mol Cancer Res. 2020 April; 18 (4): 549-559), a GST (glutathione-S-transferase)-tagged fusion BMX solution was added dropwise as a substrate protein solution to acquire a substrate having an immobilized BMX spot (diameter: about 100 μm). In order to acquire a fluorescence intensity ratio γ (described later), a GST-tagged fusion Src solution (having autophosphorylation activity similarly to BMX) was added dropwise in the same manner as above to obtain an immobilized spot on the substrate. A kinase reaction solution (25 mM Tris-HCl, 5 mM β-glycerophosphate, 0.1 mM Na₃VO₄, 10 mM MgCl₂, 1 mM ATP, and 2 mM DTT) was added, mixed, and brought into contact with each of BMX- and Src-immobilized spots and incubated at 30° C. for 120 minutes to induce an autophosphorylation reaction on the spots. Thereafter, a reaction termination solution (50 mM EDTA, 10 mM HEPES-NaOH [pH7.4], 150 mM NaCl, and 0.05% [v/v] Tween20) was brought into contact with the immobilized spots, and the mixture was incubated at 30° C. for 5 minutes to terminate the autophosphorylation reaction. After the substrate was washed with TBST, a primary antibody solution (cocktail of a mouse anti-phosphotyrosine antibody and a rabbit anti GST antibody) was added, and the mixture was incubated at 30° C. for one hour. After the substrate was washed with TBST, a secondary antibody solution (cocktail of a Goat anti-Mouse IgG (H+L) Highly Cross-Adsorbed Secondary Antibody, Alexa Fluor 790 (Invitrogen) and Goat anti-Rabbit IgG (H+L) Highly Cross-Adsorbed Secondary Antibody, Alexa Fluor 680 (Invitrogen)) was added, and the mixture was incubated at 30° C. for one hour to label the phosphorylated tyrosine appeared in the BMX immobilized spot with Alexa Fluor 790 and label a GST tag fused to BMX with Alexa Fluor 680. After labeling, the substrate was subjected to measurement by a microarray scanner having the confocal optical system to acquire a fluorescence image. Alexa Fluor 790 was measured by an excitation laser with a wavelength of 790 nm (the fluorescence signal at this time is referred to as I790 nm), and Alexa Fluor 680 was measured by an excitation laser with a wavelength of 680 nm (the fluorescence signal at this time is referred to as I680 nm). A fluorescence image having 1 pixel of 10 μm×10 μm was obtained. From the values of I790 nm and I680 nm in individual pixels, the distribution of each of the immobilized amounts of BMX and Src at individual points, to which individual pixels correspond, within spots, and the amount of phosphorylated tyrosine was acquired. In this Example, in order to reduce measurement noise, the distribution of each immobilized substrate protein amount and the distribution of phosphorylated tyrosine amount were acquired from the acquired fluorescence image after a Mean filter (radius=0.5) was applied to the fluorescence image. After the acquisition of the distributions, a moving average processing per 5 pixels was performed.

With respect to the distribution of the amount of immobilized BMX and the distribution of the amount of phosphorylated tyrosine, fitting was performed based on the Michaelis-Menten equation. In general, the Michaelis-Menten equation indicates the relationship between the concentration of a substrate and the rate of an enzyme reaction. However, the distribution of the amount of the immobilized substrate protein and the distribution of the amount of phosphorylated tyrosine acquired by the above procedure indicate the distribution of a substrate density at a point within a spot at the reaction initiation time (0 min) and the integrated amount of phosphorylated tyrosine that appears up to the end of the reaction (120 min). Because of this, in order to perform fitting in accordance with the Michaelis-Menten equation, it is necessary to consider that the substrate density decreases as the phosphorylation reaction proceeds, and simultaneously, to obtain the integrated amount of phosphorylated tyrosine up to the completion of the reaction as the time integral of the phosphorylation reaction rate. In consideration of the above, fitting in accordance with the Michaelis-Menten equation was performed based on the following (Expression 1).

$$\{ \begin{matrix}{I}_{790 nm} = {lim}_{k \to ∞} {\sum }_{i = 0}^{k - 1} \frac{{V max}_{self} {α γ S}_{i}}{{α γ S}_{i} + {Km}_{self}} Δ t + Bg \\ {α γ S}_{i + 1} = {α γ S}_{i} - \frac{{V max}_{self} {α γ S}_{i}}{{α γ S}_{i} + {Km}_{self}} Δ t \\ {S}_{o} = {I}_{680 nm} \\ {t}_{total} = k Δ t\end{matrix} \qquad Expression 1$$

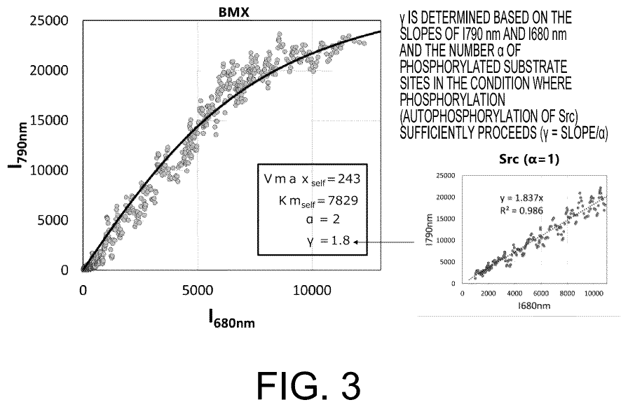

Herein, γ is a ratio of fluorescence intensity I680 nm per substrate molecule to fluorescence intensity I790 nm per phosphorylated tyrosine site, and changes under the effect of lot difference of the antibody to be used for labeling and optical conditions for photographing. In addition, a is the number of substrate sites that undergo autophosphorylation per substrate molecule. In predetermined photographing conditions, γ can be acquired by phosphorylating all the substrate proteins whose number of effective substrate sites is known, and then obtaining the slopes of fluorescence intensity I680 nm and I790 nm and the number α of effective substrate sites that undergo phosphorylation (γ=slope/α). In this Example, γ=1.8 was obtained based on the ratio 1.8 of fluorescence intensities of I680 nm and I790 nm (FIG. 3, right) in Src (under this condition, the substrate sites are sufficiently phosphorylated by autophosphorylation. The number of effective sites of the autophosphorylation reaction was considered as α=1). The number α of effective substrate sites in BMX was estimated as α=2 based on the ratio of fluorescence intensities of I680 nm and I790 nm after the substrate sites were sufficiently phosphorylated and γ=1.8. In addition, Bg, which represents the background, was set to 0 in this Example.

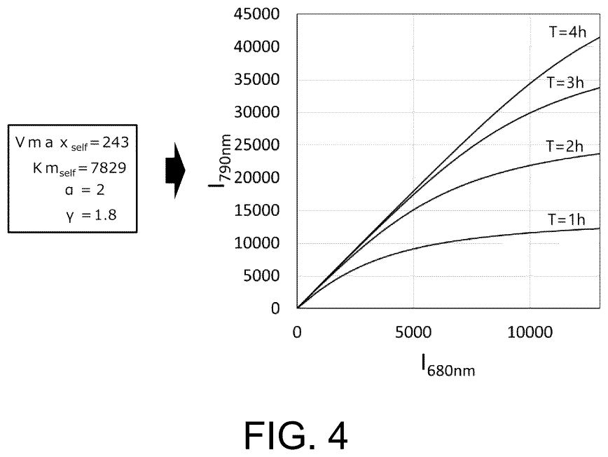

tₜₒₜₐₗ is the phosphorylation reaction time, i.e., 120 minutes. In the fitting by (Expression 1), if values of Km_{self} and Vmax_{self} are assumed, the generation rate of phosphorylated tyrosine and a decrease in the number of unreacted phosphorylation sites of BMX after a lapse of a unit time Δt at the density (I680 nm) of BMX at the initiation time of the reaction acquired above were determined. Also, based on the density of unreacted phosphorylation sites of BMX reduced in number, the generation rate of phosphorylated tyrosine at the next unit time was determined. This operation was repeated until the phosphorylation reaction time (120 minutes) was reached. In this manner, the correspondence relationship of the amount of phosphorylated tyrosine relative to the amount of immobilized BMX was estimated. Vmax_{self} and Km_{self} were acquired by converging and fitting Vmax_{self} and Km_{self} to minimize the difference between the correspondence relationship estimated herein and the correspondence relationship actually acquired of the amount of phosphorylated tyrosine relative to the amount of immobilized BMX. The left graph of FIG. 3 shows the distribution of the amount of immobilization (I680 nm) acquired for BMX and the amount of phosphorylated tyrosine (I790 nm), each point shows the values of I680 nm and I790 nm for pixel, the solid line shows the fitting curve created by the above procedure, and Vmax_{self} (/min)=243 and Km_{self}=7829 were obtained. A set of Vmax_{self} (=243), Km_{self} (=7829), α (=2), and γ (=1.8) thus acquired was stored in the memory unit as reference data. Based on the parameters acquired above and (Expression 1), it is also possible to estimate the relationship between the amount of immobilized BMX and the amount of phosphorylated tyrosine at arbitrary phosphorylation reaction time. FIG. 4 shows the result of estimating the relationship between the amount of immobilized BMX and the amount of phosphorylated tyrosine at the phosphorylation reaction times of 1 h, 3 h, and 4 h in addition to the actually measured phosphorylation reaction time of 2 hours (hereinafter, sometimes referred to as h). The estimation result thus obtained can be stored in the memory unit. As a result, even in a case where phosphorylation reaction time other than the phosphorylation reaction time set at the time of acquiring the reference data is set to measure heterophosphorylation by the specimen, the correction operation (described later) can be performed by efficiently using the reference data.

### Correction Method 1 for Autophosphorylation to Acquire Net Amount of Phosphorylated Tyrosine by Heterophosphorylation

In this Example, we will explain a case where a cell extract of human chronic myelogenous leukemia-derived cell line K562 is used as a specimen, various kinases contained in the specimen are set as kinases to be measured, and when the kinases phosphorylate, more specifically, heterophosphorylate the substrate protein BMX immobilized on a substrate, the net amount of heterophosphorylated amino acid is determined after the effect of autophosphorylation is corrected.

In this Example, we will explain a case where phosphorylated tyrosine was detected from phosphorylated amino acids, the amount of phosphorylated tyrosine was acquired as a distribution relative to the amount of the immobilized substrate, and the distribution was obtained by performing fitting according to a fitting curve based on the Michaelis-Menten equation.

An array plate having a spot of immobilized BMX (diameter: about 100 μm), which were prepared in the same procedure as in the method for acquiring reference data on autophosphorylation, and a cell extract of human chronic myelogenous leukemia-derived cell line K562 were mixed with a kinase reaction solution (25 mM Tris-HCl, 5 mM β-glycerophosphate, 0.1 mM Na₃VO₄, 10 mM MgCl₂ 1 mM ATP, and 2 mM DTT), was brought into contact with the immobilized BMX spot, and incubated at 30° C. for 120 minutes to induce a phosphorylation reaction on the spot (herein, the solutions used in the phosphorylation reaction are different only in the presence or absence of the specimen, and the other compositions are the same). Thereafter, the termination of the phosphorylation reaction, labeling, acquisition of fluorescence images, and acquisition of distributions of the amount of immobilized BMX substrate and the amount of phosphorylated tyrosine were performed in the same manner as in the method for acquiring reference data on autophosphorylation, and fitting was performed according to the following (Expression 2) similarly to (Expression 1). Since the distribution of the amount of phosphorylated tyrosine relative to the acquired amount of immobilized substrate occurs by both heterophosphorylation and autophosphorylation reaction, the maximum reaction rate, the Michaelis constant, and the number of effective phosphorylation sites are represented as Vmaxₜₒₜₐₗ, Kmₜₒₜₐₗ, and αₜₒₜₐₗ, respectively.

$$\{ \begin{matrix}{I}_{790 nm} = {lim}_{k \to ∞} {\sum }_{i = 0}^{k - 1} \frac{{V max}_{total} {α}_{total} {γ S}_{i}}{{α}_{total} {γ S}_{i} + {Km}_{total}} Δ t + Bg \\ {α}_{total} {γ S}_{i + 1} = {α}_{total} {γ S}_{i} - \frac{{V max}_{total} {α}_{total} {γ S}_{i}}{{α}_{total} {γ S}_{i} + {Km}_{total}} Δ t \\ {S}_{o} = {I}_{680 nm} \\ {t}_{total} = k Δ t\end{matrix} \qquad Expression 2$$

Herein, γ was set to γ=1.8 similarly in the method for acquiring reference data on autophosphorylation, and the number of all effective sites αₜₒₜₐₗ of phosphorylation including heterophosphorylation and autophosphorylation was set to αₜₒₜₐₗ=4 since the number of tyrosine phosphorylation sites of BMX reported in PhosphoSitePlus is 4 (note that, as αₜₒₜₐₗ, the value reported in PhosphoSitePlus may be used. Other than this, αₜₒₜₐₗ may be an actual measurement value obtained from the ratio of the fluorescence intensity of I790 nm and the fluorescence intensity of I680 nm and γ when the phosphorylation reaction with the specimen is performed for a period longer than 120 minutes to phosphorylate all the effective phosphorylation sites). In addition, Bg=1634 was employed. As a result of fitting, the fitting curve where Vmaxₜₒₜₐₗ=403 and Kmₜₒₜₐₗ=15957 was obtained, as illustrated in FIG. 4. FIG. 4 shows the distribution of immobilization amount (I680 nm) acquired for BMX and the amount of phosphorylated tyrosine (I790 nm) in a case of a reaction with a K562 cell extract, and each point shows the values of 1680 nm and 1790 nm per pixel, and the solid line is the fitting curve created by the above procedure.

The measurement results of FIG. 4 has an effect of autophosphorylation by BMX itself in addition to heterophosphorylation caused by a kinase in the K562 cell extract. Then, in this Example, as correction method 1, the amount of phosphorylated tyrosine generated by autophosphorylation was subtracted as a background from the measured amount of phosphorylated tyrosine (I790 nm) for each amount of immobilized BMX (FIG. 6). For the distribution of the amount of phosphorylated tyrosine to the amount of immobilization generated by autophosphorylation, the data in storage acquired according to the procedure of the method for acquiring reference data regarding autophosphorylation was used by reference from the memory unit. As a result, it is possible to acquire a net amount of heterophosphorylation excluding the amount of phosphorylated tyrosine excessively generated by autophosphorylation.

In this Example, in performing correction method 1, competition between autophosphorylation and heterophosphorylation for substrate sites is not considered. Since the amount of phosphorylated amino acid when phosphorylation was performed only by autophosphorylation is larger than the amount of phosphorylated amino acid generated by autophosphorylation when phosphorylation is performed by autophosphorylation and heterophosphorylation, the amount of phosphorylated amino acid by autophosphorylation is estimated excessively and that by heterophosphorylation is estimated slightly low.

### Correction Method 2 for Autophosphorylation for Acquiring Net Amount of Phosphorylated Tyrosine by Heterophosphorylation

We will describe Correction method 2, which is made, similarly to correction method 1, for the purpose of acquiring a net amount of phosphorylated tyrosine by heterophosphorylation when a substrate protein immobilized on a substrate is phosphorylated by kinases contained in a specimen, in other words, heterologous phosphorylated, and performed in consideration of competition between autophosphorylation and heterophosphorylation for substrate sites.

For expressing the relationship between the amount of immobilized substrate and the amount of phosphorylated amino acid when autophosphorylation and heterophosphorylation are competitively made, the following expression (Expression 3) was conceived.

$$\{ \begin{matrix}{I}_{790 nm} = {lim}_{k \to ∞} {\sum }_{i = 0}^{k - 1} Pi + Bg \\ {P}_{i} = {P}_{i - 1} + \{ \frac{{V max}_{self} {Site}_{i - 3 common}}{{Km}_{self} + {Site}_{i - 3 common}} + \frac{{V max}_{other} ( {Site}_{i - 1 common} + {Site}_{i - 1 other} )}{{Km}_{other} + ( {Site}_{i - 1 common} + {Site}_{i - 1 other} )} \} Δ t \\ {Site}_{i common} = {Site}_{i - 1 common} - \{ \frac{{V max}_{self} {Site}_{i - 1 common}}{{Km}_{self} + {Site}_{i - 1 common}} + \frac{{Site}_{i - 1 common}}{{Site}_{i - 1 common} + {Site}_{i - 1 other}} \times \frac{{V max}_{other} ( {Site}_{i - 1 common} + {Site}_{i - 1 other} )}{{Km}_{other} + ( {Site}_{i - 1 common} + {Site}_{i - 1 other} )} \} Δ t \\ {Site}_{i other} = {Site}_{i - 1 other} - \{ \frac{{Site}_{i - 1 other}}{{Site}_{i - 1 common} + {Site}_{i - 1 other}} \times \frac{{V max}_{other} ( {Site}_{i - 1 common} + {Site}_{i - 1 other} )}{{Km}_{other} + ( {Site}_{i - 1 common} + {Site}_{i - 1 other} )} \} Δ t \\ {Site}_{0 common} = {α γ I}_{680 nm} \\ {Site}_{i other} = ( {α}_{total} - α ) {γ I}_{680 nm} \\ {t}_{total} = k Δ t\end{matrix} \qquad Expression 3$$

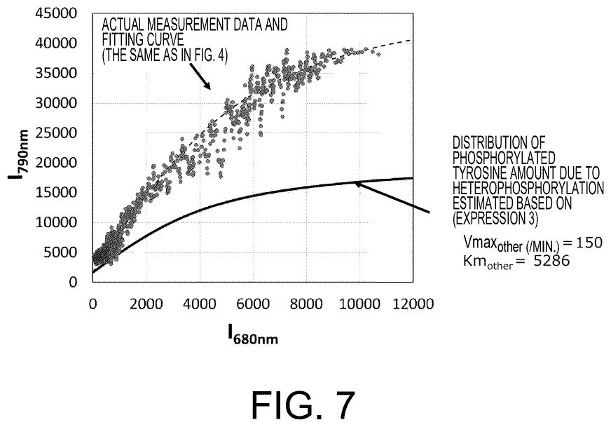

In (Expression 3), Vmax_{self} and Km_{self} represent the maximum reaction rate and Michaelis constant of autophosphorylation, respectively. Vmaxₒₜₕₑᵣ and Kmₒₜₕₑᵣ are the maximum reaction rate and Michaelis constant of heterophosphorylation, respectively. Note that, I790 nm and I680 nm are fluorescence intensities indicating the amount of immobilized substrate and the amount of phosphorylated tyrosine in a substrate spot acquired using the same procedure as in correction method 1 for autophosphorylation for acquiring a net amount of phosphorylated tyrosine by heterophosphorylation. Bg is a background when I790 nm is measured. Reference symbol α is the number of substrate sites (equal to the number of substrate sites in the case of autophosphorylation) for which an autophosphorylation reaction and a heterophosphorylation reaction competitively occur per substrate protein molecule, and αₜₒₜₐₗ is the total number of substrate sites phosphorylated per substrate protein molecule regardless of autophosphorylation and heterophosphorylation per substrate protein molecule. The reference symbol Tₜₒₜₐₗ is the phosphorylation reaction time when the measurement is performed on the specimen, and γ is the ratio of the fluorescence intensity of I680 nm per molecule of substrate and the fluorescence intensity of I790 nm per site of phosphorylated tyrosine when the measurement is performed on the specimen. The reference symbol Site_{i common} is the number of unreacted substrate sites in a case where an autophosphorylation reaction and a heterophosphorylation reaction competitively occur at time i, and Site_{i other} is the number of unreacted substrate sites in a case where a heterophosphorylation reaction alone occurs at time i. Similarly to correction method 1 for autophosphorylation in order to acquire a net amount of phosphorylated tyrosine by heterophosphorylation, a cell extract of human chronic myelogenous leukemia-derived cell line K562 was used as a specimen, and the phosphorylation reaction, termination reaction, labeling, and optical measurement were performed on an immobilization spot of BMX to acquire the distribution of the amount of immobilization (I680 nm), and then the amount of phosphorylated tyrosine (I790 nm), and the measurement data was fitted by fitting based on (Expression 3). Vmaxₒₜₕₑᵣ and Kmₒₜₕₑᵣ in the case of heterophosphorylation were determined. As a result, Vmaxₒₜₕₑᵣ=150 and Kmₒₜₕₑᵣ=5286 were obtained, and the distribution of the net amount of phosphorylated tyrosine due to heterophosphorylation was acquired, as illustrated in FIG. 7.

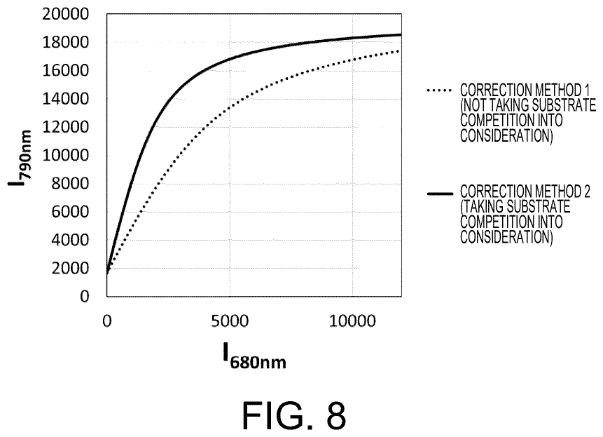

The distributions of the amounts of phosphorylated tyrosine due to heterophosphorylation obtained by correction method 1 and correction method 2 were compared. The results are shown in FIG. 8. In correction method 1, since competition of autophosphorylation and heterophosphorylation at a substrate site is not taken into consideration, autophosphorylation was estimated slightly excessively, with the result that the amount of phosphorylated amino acid by heterophosphorylation was estimated to be low. In contrast, in correction method 2, since competition of autophosphorylation and heterophosphorylation at a substrate site is taken into consideration, the resulting amount of heterophosphorylation was estimated to be larger than that in correction method 1 in any amount of immobilization.

At least one of the embodiments described above allows evaluation of kinase activity while correcting for the effect of autophosphorylation of the substrate protein itself.

While certain embodiments have been described, these embodiments have been presented by way of example only and are not intended to limit the scope of the inventions. The embodiments may be in a variety of other forms. Furthermore, various omissions, substitutions and changes may be made without departing from the spirit of the inventions. The embodiments and their modifications are included in the scope and the subject matter of the invention, and at the same time included in the scope of the claimed inventions and their equivalents.

1. An apparatus for assessing kinase activity comprising:
a processing circuitry configured to
measure an amount of phosphorylated amino acid in immobilized substrate protein generated when the substrate protein is reacted with a kinase to be evaluated, as an amount of phosphorylated amino acid in a specimen,
read, from a memory, information which links reaction time, an amount of immobilized substrate protein having autophosphorylation activity, and an amount of autophosphorylated amino acid generated in the substrate protein having autophosphorylation activity, and correct the amount of phosphorylated amino acid in the specimen based on a measurement result for the specimen and the information; and
evaluate kinase activity based on the corrected amount of phosphorylated amino acid in the specimen.

2. The apparatus of claim 1, further comprising the memory.

3. The apparatus of claim 1,
wherein the correction reads the information from the memory connected via a network.

4. The apparatus of claim 1,
wherein the information comprises one or two or more selected from the group consisting of the number of effective phosphorylation sites, maximum reaction rate (Vmax), and Michaelis constant (Km) of the substrate protein having autophosphorylation activity.

5. The apparatus of claim 4,
wherein the number of effective phosphorylation sites comprises the number of all effective phosphorylation sites (αₜₒₜₐₗ) of a substrate protein having autophosphorylation activity, or the number of phosphorylation sites (α) that undergo not only a heterophosphorylation reaction but also an autophosphorylation reaction.

6. The apparatus of claim 4,
wherein the maximum reaction rate (Vmax) comprises a maximum reaction rate (Vmax_{self}) of an autophosphorylation reaction at the phosphorylation sites that undergo not only a heterophosphorylation reaction but also an autophosphorylation reaction of a substrate protein having autophosphorylation activity.

7. The apparatus of claim 4,
wherein the Michaelis constant (Km) comprises Michaelis constant (Km_{self}) of an autophosphorylation reaction at the phosphorylation sites that undergo not only a heterophosphorylation reaction but also an autophosphorylation reaction of a substrate protein having autophosphorylation activity.

8. The apparatus of claim 1,
wherein the correction corrects the amount of phosphorylated amino acid in the specimen by subtracting or removing the amount of autophosphorylated amino acid from the amount of phosphorylated amino acid in the specimen, as a measurement result for the specimen.

9. The apparatus of claim 1, wherein the processing circuitry is further configured to control an output unit so as to output an evaluation result of kinase activity.

10. A non-transitory computer readable storage medium, which stores a program that causes an apparatus for assessing kinase activity to execute processing comprising:
measuring an amount of phosphorylated amino acid in immobilized substrate protein generated when the substrate protein is reacted with a kinase to be evaluated, as an amount of phosphorylated amino acid in a specimen,
reading, from a memory, information which links reaction time, an amount of immobilized substrate protein having autophosphorylation activity, and an amount of autophosphorylated amino acid generated in the substrate protein having autophosphorylation activity, and correcting the amount of phosphorylated amino acid in the specimen based on a measurement result for the specimen and the information; and
evaluating kinase activity based on the corrected amount of phosphorylated amino acid in the specimen.

11. The non-transitory computer readable storage medium according to claim 10,
wherein the non-transitory computer readable storage medium stores the information which links reaction time, an amount of immobilized substrate protein having autophosphorylation activity, and an amount of autophosphorylated amino acid generated in the substrate protein having autophosphorylation activity.

12. The non-transitory computer readable storage medium according to claim 10,
wherein the correcting further comprises reading the information from the memory connected via a network.

13. The non-transitory computer readable storage medium according to claim 10,
wherein the information comprises one or two or more selected from the group consisting of the number of effective phosphorylation sites, maximum reaction rate (Vmax), and Michaelis constant (Km) of the substrate protein having autophosphorylation activity.

14. The non-transitory computer readable storage medium according to claim 13,
wherein the number of effective phosphorylation sites comprises the number of all effective phosphorylation sites (αₜₒₜₐₗ) of a substrate protein having autophosphorylation activity, or the number of phosphorylation sites (α) that undergo not only a heterophosphorylation reaction but also an autophosphorylation reaction.

15. The non-transitory computer readable storage medium according to claim 13,
wherein the maximum reaction rate (Vmax) comprises a maximum reaction rate (Vmax_{self}) of an autophosphorylation reaction at the phosphorylation sites that undergo not only a heterophosphorylation reaction but also an autophosphorylation reaction of a substrate protein having autophosphorylation activity.

16. The non-transitory computer readable storage medium according to claim 13,
wherein the Michaelis constant (Km) comprises Michaelis constant (Km_{self}) of an autophosphorylation reaction at the phosphorylation sites that undergo not only a heterophosphorylation reaction but also an autophosphorylation reaction of a substrate protein having autophosphorylation activity.

17. The non-transitory computer readable storage medium according to claim 10,
wherein the correcting further correct the amount of phosphorylated amino acid in the specimen by subtracting or removing the amount of autophosphorylated amino acid from the amount of phosphorylated amino acid in the specimen, as a measurement result for the specimen.

18. The non-transitory computer readable storage medium according to claim 10, further comprising:
controlling an output unit so as to output an evaluation result of kinase activity by the processing circuitry.
